(19) United States
(12) Plant Patent Application Publication
(10) Pub. No.: US 2005/0114973 P1
(43) Pub. Date: May 26, 2005

(54) Zoysiagrass plant 'TM9'

(75) Inventor: Kunio Matsui, Nishikamo-gun (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: TOYOTA JIDOSHA KABUSHIKI KAISHA, Toyota-shi (JP)

(21) Appl. No.: 10/936,495

(22) Filed: Sep. 9, 2004

(30) Foreign Application Priority Data
Nov. 21, 2003 (JP) 16314

(51) Int. Cl. A01H 5/00

(52) U.S. Cl. PLT/390

(57) ABSTRACT

The present culivar TM9 (Zoysia matrella Merr.) advantageously has a growing rate of in terms of plant height less than half as compared with existing cultivars and requires mowing every 20 to 40 days during the summer so as to be maintained in fair condition and allows the amount of fertilizer to be reduced to almost less than half.

### LATIN NAME OF THE GENUS AND SPECIES OF THE PLANT CLAIMED

The present invention relates to the species Zoysia matrella Merr.

Variety denomination: ‘TM9’.

### BACKGROUND OF THE INVENTION

### Field of Invention

The present invention relates to a new and distinct perennial variety of Zoysiagrass asexually reproduced and selected.

### SUMMARY OF THE INVENTION

The present invention relates to a new and distinct Zoysiagrass cultivar (Zoysia matrella Merr.) named ‘TM9’.

Zoysiagrass is a popular lawn grass which has been widely used as greening turf. The turf thereof, however, should be mown every 7 to 10 days during the summer so as to be maintained in beautiful condition. Since the newly developed TM9 advantageously has a growing rate in terms of plant height less than half as compared with conventional cultivars, mowing is required every 20 to 40 days during the summer to maintain fair condition and the amount of fertilizer can be reduced to almost less than half. Therefore, labors and cost necessary for maintenance of the turf as well as clipping from lawn-mowing can be reduced. Furthermore, the cultivar can be readily grown in a place where a lawnmower cannot be conveniently used, such as on a rooftop of a building.

In addition, it is expected that prevalence of TM9 facilitates greening of the rooftop and schoolyard and leads to alleviation of heat island phenomenon and reduction of the amount of CO₂.

For purposes of registration under the “International Convention for the Protection of New Varieties of Plants” (generally known by its French acronym as the UPOV Convention) and noting sections 1612 of the Manual of Patent Examining Procedure, it is proposed that the new variety of Zoysiagrass of the present invention be named ‘TM9’.

### BRIEF DESCRIPTIONS OF THE ILLUSTRATIONS

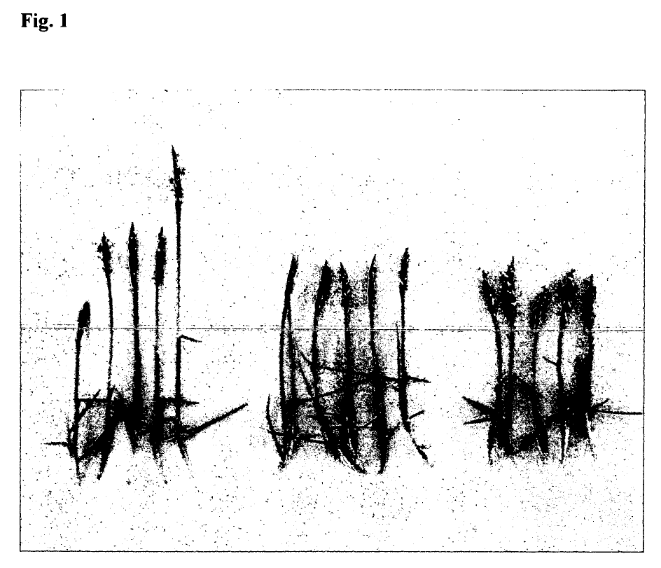

FIG. 1 is a set of photographs comparing spikestems of control cultivars and the present cultivar (TM9) (Left: Emerald; Middle: Winter Field; Right: TM9). Photographed on May 13, 2003 at Toyota Biotechnology & Afforestation Laboratory (located in Nishikamo-gun in Aichi Prefecture, Japan).

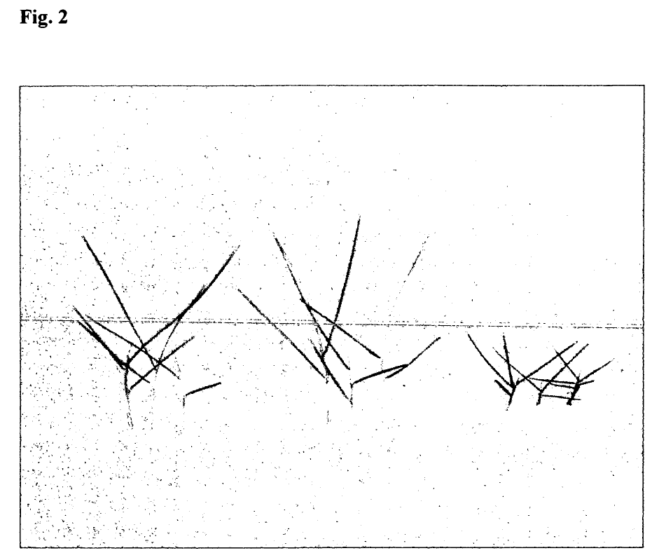

FIG. 2 is a set of photographs comparing leaf blades of control cultivars and the present cultivar (TM9) (Left: Emerald; Middle: Winter Field; Right: TM9). Photographed on Jul. 15, 2003 at Toyota Biotechnology & Afforestation Laboratory (located in Nishikamo-gun in Aichi Prefecture, Japan).

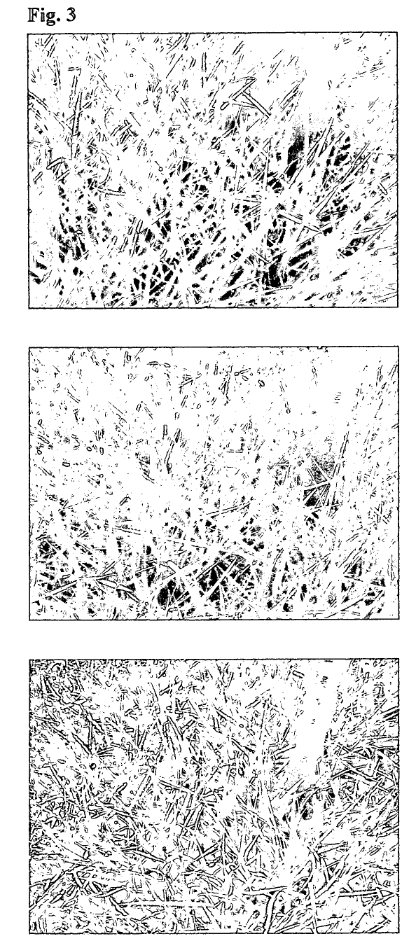

FIG. 3 is a set of photographs comparing grass shapes of control cultivars and the present cultivar (TM9) (Upper: Emerald; Middle: Winter Field; Lower: TM9). Photographed on Jul. 15, 2003 at Toyota Biotechnology & Afforestation Laboratory (located in Nishikamo-gun in Aichi Prefecture, Japan).

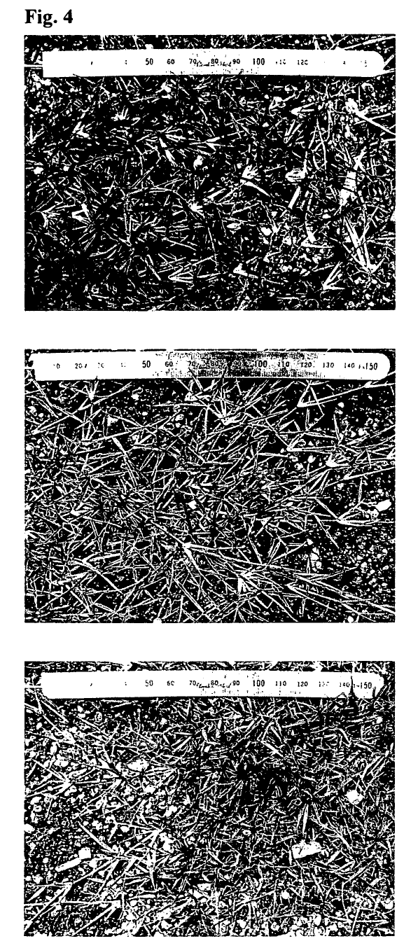

FIG. 4 is a set of photographs comparing stolons of control cultivars and the present cultivar (TM9) (Upper: Emerald; Middle: Winter Field; Lower: TM9). Photographed on Jul. 15, 2003 at Toyota Biotechnology & Afforestation Laboratory (located in Nishikamo-gun in Aichi Prefecture, Japan).

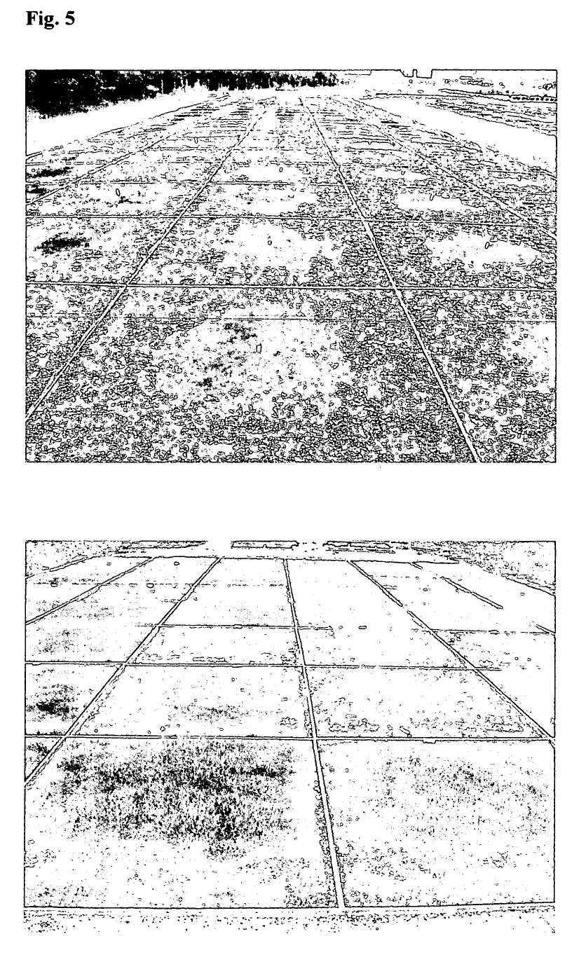

FIG. 5 is a set of photographs showing full views of test field for the present cultivar (TM9) (Upper: Individually planted plot; Lower: Densely plated plot). Photographed on Jul. 15, 2003 at Toyota Biotechnology & Afforestation Laboratory (located in Nishikamo-gun in Aichi Prefecture, Japan).

### DETAILED BOTANICAL DESCRIPTION OF THE PLANT

1. Characteristics of the plant body of the present invention

1) Overview

Fifty thousand seeds of conventional Zoysiagrass cultivars were irradiated with soft X-ray (in the year 2000), 500 individuals exhibiting short plant height as a pot seedling were selected from the germinated individuals (in the year 2001), and 100 individuals were selected from among these seedlings based on their growing properties as a pot seedling. They were planted in a field and an outdoor cultivation test was initiated (in the year 2002). The present cultivar was obtained after completion of cultivation tests in various locations throughout Japan (completed in the year 2004). Characteristics of the present cultivar are as follows: creeping plant form; spikestem of medium-sized thickness and short length; extremely dense stolons of medium-sized thickness; leaves of very short length, narrow width and a thick color; a small number of spikelets; moderate initial growth; moderate plant vigor in spring and fall; medium (not early nor late) timing in spike-sprouting; spike-sprouting only in spring with a large number of spikes; medium (not early nor late) timing in turning green; medium (not early nor late) timing in turning red; good winter hardiness; good summer tolerance; medium shade tolerance; medium drought tolerance; medium salinity tolerance; medium tread pressure resistance.

2) Comparison with control cultivars

| | | | Present | | |
|---|---|---|---|---|---|
| | | | cultivar | Control cultivar | |
| | Item | Trait | TM9 | Emerald | Winter Field |
| 1 | Plant form | Plant form | Creeping | Intermediate | Intermediate |
| 2 | Stem | Thickness | Medium | Medium | Medium |
| | | of | (0.9 mm) | (0.9 mm) | (0.9 mm) |
| | | spikestem | | | |
| | | Length of | Short (3 cm) | Short (3 cm) | Short (3 cm) |
| | | spikestem | | | |
| 3 | Stolon | Density of | Extremely | Medium to | Medium to |
| | | stolons | dense | dense | dense |
| | | Thickness | Medium | Fine to | Fine to |
| | | of stolon | (1.3 mm) | Medium | Medium |
| | | | | (1.1 mm) | (1.0 mm) |
| 4 | Foliage | Leaf | Extremely | Short (6 cm) | Short to |
| | | length | short | | Medium |
| | | | (3 cm) | | (10 cm) |
| | | Leaf width | Narrow | Narrow | Narrow |
| | | | (1.6 mm) | (1.7 mm) | (1.6 mm) |
| | | Leaf color | Thick | Medium | Medium |
| 5 | Spike | Spike | Short | Short | Short |
| | | length | (13 mm) | (14 mm) | (12 mm) |
| | | Spike | Thick | Thick | Thick |
| | | color | | | |
| | | Spikelet | Medium | Medium | Medium |
| | | length | | | |
| | | Spikelet | Medium | Medium | Medium |
| | | width | | | |
| | | Number of | Few | Few | Few |
| | | spikelet | | | |
| 6 | Initial | Initial | Medium | Medium | Medium |
| | growth | growth | | | |
| 7 | Plant vigor | Plant | Moderate | Moderate | Moderate |
| | | vigor in | | | |
| | | spring | | | |
| | | Plant | Moderate | Good | Good |
| | | vigor in | | | |
| | | fall | | | |
| 8 | Spike | Beginning | Medium | Medium | Medium |
| | -sprouting | of spike - | (not early | (not early | (not early |
| | time | sprouting | nor late) | nor late) | nor late) |
| 9 | Spike | Spike - | Spike | Spike | Spike |
| | -sprouting | sprouting | -sprouting | -sprouting | -sprouting |
| | properties | in | only in | only in | only in |
| | | spring/fall | spring | spring | spring |
| | | Number of | Many | Medium | Medium |
| | | spikes | | | |
| 10 | Growing | Timing to | Medium | Medium | Medium |
| | period | turn green | (not early | (not early | (not early |
| | | | nor late) | nor late) | nor late) |
| | | Timing to | Medium | Medium | Medium |
| | | turn red | (not early | (not early | (not early |
| | | | nor late) | nor late) | nor late) |
| 11 | Winter | Good/Bad | good | good | good |
| | hardiness | in winter | | | |
| | | hardiness | | | |
| 12 | Summer | Good/Bad | good | good | good |
| | tolerance | in summer | | | |
| | | tolerance | | | |
| 13 | Environ- | Shade | Medium | Medium | Medium |
| | mental | tolerance | | | |
| | | tolerance | | | |
| | | Drought | Medium | Medium | Medium |
| | | tolerance | | | |
| | | Salinity | Medium | Medium | Medium |
| | | tolerance | | | |
| 14 | Tread | Tread | Medium | Medium | Medium |
| | pressure | pressure | | | |
| | resistance | resistance | | | |

* Notes:
Type of plant: lawn
Name of the applied cultivar: TM9
Name or appellation of the assignee: Toyota Motor Corporation
Inventor: Kunio Matsui
Address of the assignee: 1 Toyota-Cho, Toyota City, Aichi Prefecture, Japan
Cultivation site: Toyota Biotechnology & Afforestation Laboratory Nishikamo-gun in Aichi Prefecture, Japan
Place where characteristics research was conducted: Toyota Biotechnology & Afforestation Laboratory, Nishikamo-gun in Aichi Prefecture, Japan
Name of researcher who conducted characteristics research: Kunio Matsui
Period when characteristics research was conducted: 2002-2003
Control cultivar (most approximate cultivar): Emerald

3) Characteristics by which the present cultivar is distinguished from the control cultivars
a) Control cultivars

Emerald

Winter Field

b) Distinguishing characteristics

The present cultivar can be distinguished from Emerald in that the present cultivar has a creeping plant form (wheareas Emerald has an intermediate, i.e. semi-errect or semi-creeping plant form); extremely dense stolons (whereas Emerald has a medium to low stolon density); leaves of very short length (wheareas Emerald has short leaves) and a thick color (wheareas Emerald has a medium leaf color); moderate plant vigor in fall (wheareas Emerald has a good plant vigor); and a large number of spikes (wheareas Emerald has a medium number of spikes).

The present cultivar can be distinguished from Winter Field in that the present cultivar has a creeping plant form (wheareas Winter Field has an intermediate, i.e. semi-errect or semi-creeping plant form); extremely dense stolons (whereas Winter Field has a medium to low stolon densisity); leaves of very short length (wheareas Winter Field has short leaves) and a thick color (wheareas Winter Field has a medium leaf color); moderate plant vigor in fall (wheareas Winter Field has a good plant vigor); and a large number of spikes (wheareas Winter Field has a medium number of spikes).

4) Cultivation conditions for characteristic research experiment of above 1) to 3)

a Cultivation site: Toyota Biotechnology & Afforestation Laboratory Nishikamo-gun in Aichi Prefecture, Japan

b Cultivation time and period: 20 months from April, 2002

c Cultivation method (Examples: cultivation style such as on bare ground, in a facility, field planting or pot planting, cultivation scale, etc.)

Pot seedlings reproduced by root separating were raised in greenhouse for two months and fix planted on bare ground in April along with control cultivars in order to perform a characteristic research experiment. Five individuals (triplicates) were fix planted at a density of one seedling per an area of 200 cm×200cm in an individually planted plot while they were fix planted in triplicates at a density of one seedling per an area of 15 cm×15cm in a dense plated test plot having an area of 150 cm×150 cm thereby performing a characteristic research experiment.

2. Reproduction method

Vegetative propagation

3. Conditions of keeping and storage the plant having identifiable characteristics

Place where the plant is maintained and/or stored: Toyota Biotechnology & Afforestation Laboratory (located in Nishikamo-gun in Aichi Prefecture, Japan)

Method of maintenance/storage: pot planting and ground planting

4. History of cultivating the present cultivar

1) Material of new cultivar

Mother: conventional cultivar(marketed cultivar cultivated in Ibaraki Prefecture, Japan)

Father —

Family tree —

2) Cultivation site

Toyota Biotechnology & Afforestation Laboratory (located in Nishikamo-gun in Aichi Prefecture, Japan)

3) History of cultivating the cultivar

Fifty thousand self-fertile seeds of conventional Zoysiagrass cultivars (raised in Ibaraki Prefecture, Japan) were irradiated with soft X-ray in September, 2000, and individuals exhibiting short plant height as a pot seedling were selected from the germinated individuals. The thus selected lines were reproduced by root separation in May, 2001 and the uniformity of these vegetative propagated lines was respectively confirmed in greenhouse by February, 2002. Newly root separated lines were fix planted in a field in April, 2002. A line exhibiting a short leaf length and a satisfactory initial growth was selected and the stability thereof was confirmed to complete the cultivating of the new cultivar on 20 Nov., 2003.

5. Main use of the present cultivar

The present cultivar can be used for ground covering of a park, garden, etc.

6. Other items in relation to the cultivation of the present cultivar

1) Applicable area

Warm area

2) Specific cultivation site in Japan

Address (Zip code: 470-0201):

Toyota Biotechnology & Afforestation Laboratory

No. 1099 Kurozasa-Marune, Miyoshi-machi, Nishikamo-gun, Aichi Prefecture, Japan

Facility for travel: Meitetsu Toyoda line (Closest station: Miyoshigaoka)

3) Cultivation style: Normal cultivation

Bare ground

Seasons suitable for seeding, planting, etc.

Other reproduction method:

Vegetative propagation by stolons from the beginning of April to the beginning of May/year round

Blooming season, harvest season, and any other seasons in cultivation stage suitable for specifying characteristics of the present cultivar

Blooming season: from the beginning to the end of May/year round

Greening season: from the end of April to the end of October/year round

4) Other items to be mentioned for the cultivation of the present cultivar

The present cultivar is creeping in plant form and short in leaf length, which allows weeds readily to grow. Frequent weeding is therefore required.

5) Information about the cultivation technique and raising condition of the present cultivar is available from the below:

Address (Zip code: 470-0201):

Toyota Biotechnology & Afforestation Laboratory

No. 1099 Kurozasa-Marune, Miyoshi-machi, Nishikamo-gun, Aichi Prefecture, Japan

Person in charge belongs to Biotechnology & Afforestation Business Division (TEL 0561-36-8441)

Name: Kunio Matsui

7. Other Information

When the present cultivar is allowed freely to grow, the plant height (i.e. the height from the ground to the tip of the leaf blade) is as about half as that of Emerald.

1. A new and distinct variey of Zoysia matrella Merr. named TM9 illustrated and described in the present invention.
